(19) United States
(12) Patent Application Publication
(10) Pub. No.: US 2023/0322662 A1
(43) Pub. Date: Oct. 12, 2023

(54) ALKANE MULTI-SULFONIC ACIDS, COMPOSITIONS THEREOF, AND RELATED METHODS

(71) Applicant: University of Kansas, Lawrence, KS (US)

(72) Inventors: Mark Brandon Shiflett, Lawrence, KS (US); Rajkumar Kore, Lawrence, KS (US); Aaron M. Scurto, Oskaloosa, KS (US)

(21) Appl. No.: 18/042,528

(22) PCT Filed: Aug. 20, 2021
(86) PCT No.: PCT/US21/46857

§ 371 Date: Feb. 22, 2023

Related U.S. Application Data
(60) Provisional application No. 63/069,318, filed on Aug. 24, 2020.

(51) Int. Cl. C07C 309/05; C07C 303/06; C07C 2/86

(52) U.S. Cl. CPC C07C 309/05; C07C 303/06; C07C 2/868

(57) ABSTRACT

Alkane multi-sulfonic acids and salts thereof are provided. In embodiments, an alkane multi-sulfonic acid or salt thereof comprises an alkyl group and at least two sulfonic acid groups, the alkane multi-sulfonic acid having a total number of carbon atoms of from 2 to 9, wherein the alkane multi-sulfonic acid does not comprise a halogen.

### CROSS-REFERENCE TO RELATED APPLICATIONS

The present application claims priority to U.S. provisional patent application No. 63/069,318 that was filed Aug. 24, 2020, the entire contents of which are incorporated herein by reference.

### BACKGROUND

Sulfuric acid (H₂SO₄), a strong acid, finds use in many applications, e.g., catalyzing the conversion of cyclohexanone oxime to caprolactam (an intermediate in the production of nylon) to catalyzing the alkylation of isobutane in the production of motor fuel. Fluoroalkane sulfonic acids have been developed in order to increase the acidity of sulfuric acid. The processes used to synthesize the fluoroalkane sulfonic acids require electrochemistry, increasing the cost and limiting the availability of such acids.

### SUMMARY

The present disclosure provides alkane multi-sulfonic acids and processes for their preparation. Ionic liquids and catalyst compositions based on the alkane multi-sulfonic acids are also provided, as well as applications for the alkane multi-sulfonic acids and compositions thereof.

Alkane multi-sulfonic acids and salts thereof are provided. In embodiments, an alkane multi-sulfonic acid or salt thereof comprises an alkyl group and at least two sulfonic acid groups, the alkane multi-sulfonic acid having a total number of carbon atoms of from 2 to 9, wherein the alkane multi-sulfonic acid does not comprise a halogen. Processes for making and using the alkane multi-sulfonic acids and salts thereof are also provided.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A-1C show illustrative cations which may be used to form an ionic liquid for use in catalyst compositions comprising the present alkane multi-sulfonic acids or for forming an ionic liquid from the present alkane multi-sulfonic acids.

FIG. 1D shows illustrative cations which may be used to form an ionic liquid for use in catalyst compositions comprising the present alkane multi-sulfonic acids or for forming an ionic liquid from the present alkane multi-sulfonic acids.

FIG. 1E shows illustrative bases which may be combined with the present alkane multi-sulfonic acids to form an ionic liquid.

FIG. 2 shows illustrative anions which may be used to form an ionic liquid for use in catalyst compositions comprising the alkane multi-sulfonic acids.

FIG. 3 shows illustrative aromatics for use in catalyst compositions comprising the present alkane multi-sulfonic acids.

FIG. 4 is a schematic of a one-step process which may be used to prepare the present alkane multi-sulfonic acids, using tetrachloroethene and its conversion to ethane-1,1,2,2-tetrasulfonic acid as an illustrative example.

### DETAILED DESCRIPTION

The present disclosure provides alkane multi-sulfonic acids and processes for their preparation. Compositions based on the alkane multi-sulfonic acids are also provided, as well as applications for the alkane multi-sulfonic acids and compositions thereof. The present alkane multi-sulfonic acids span a wide range of acidity and solubility, rendering them useful for a variety of applications requiring an acid. Single-step processes of making the alkane multi-sulfonic acids are also provided, which are more simple and cheaper than existing processes of making fluoroalkane sulfonic acids. The present alkane multi-sulfonic acids may also be used to form ionic liquids with advantageous properties related to the alkane multi-sulfonate anion component, e.g., as compared to existing halogenated anions. The resulting ionic liquids may be used in a variety of applications requiring an ionic liquid.

Alkane Multi-Sulfonic Acids

The present disclosure provides certain alkane multi-sulfonic acid compounds. Although one or more provisos may apply, in general, the “alkane multi-sulfonic acid” comprises an alkyl group and two or more sulfonic acid groups. No halogen atoms are present in the alkane multi-sulfonic acids. The alkyl group may have from 1 to 9 carbon atoms, i.e., the alkyl group may be a methyl, ethyl, propyl, butyl, etc. This encompasses alkyl groups having 2, 3, 4, 5, 6, 7, 8, or 9 carbon atoms, i.e., the alkyl group may be an ethyl, propyl, butyl, etc. The alkyl group may be linear, cyclic, or branched. The sulfonic acid groups (—SO₃H) are covalently bound to carbon atoms of the alkyl group, although more than one sulfonic acid group may be bound to the same carbon atom. In embodiments, the number of sulfonic acid groups in the alkane multi-sulfonic acid is two, three, four, etc.

In embodiments, the alkane multi-sulfonic acid comprises an alkyl group selected from methyl, ethyl, propyl, and butyl; and two, three, or four sulfonic acid groups; wherein no halogen atoms are present. In this embodiment, variations may apply such as one or more of: the alkyl group is selected from ethyl, propyl, and butyl; and two, three, or four SO₃H are present.

In embodiments, the alkane multi-sulfonic acid has the formula CR₃—CR₂—SO₃H, wherein at least one R is SO₃H; each remaining R is independently selected from hydrogen, CₙH₍₂ₙ₊₁₎, CₙH₍₂ₙ₋₁₎, and SO₃H; n is from 0 to 7; and the alkane sulfonic acid has a total number of carbon atoms from 2 to 9. In further embodiments, the formula is CR₂(SO₃H)—CR₂—SO₃H, wherein each R is independently selected from hydrogen, CₙH₍₂ₙ₊₁₎, CₙH₍₂ₙ₋₁₎, and SO₃H; n is from 0 to 7; and the alkane sulfonic acid has a total number of carbon atoms from 2 to 9. The formula CₙH₍₂ₙ₊₁₎ encompasses both linear and branched structures while the formula CₙH₍₂ₙ₋₁₎ encompasses cyclic structures. In these embodiments, one or more provisos may apply such as: each remaining R is independently selected from hydrogen, CₙH₍₂ₙ₊₁₎ and SO₃H; n is 0, 1, or 2; the total number of carbons is from 2 to 4; at least three SO₃H are present; at least four SO₃H are present; and two, three, or four SO₃H are present.

In embodiments, the alkane multi-sulfonic acid has the formula CₙH₍₂ₙ₊₁₎—CR₂—CR₂—SO₃H, wherein n is from 0 to 7; at least one R is SO₃H; and each remaining R is independently selected from hydrogen and SO₃H. In further embodiments, the formula is CₙH₍₂ₙ₊₁₎—CR(SO₃H)—CR₂—SO₃H, wherein n is from 0 to 7 and each R is independently selected from hydrogen and SO₃H. In these embodiments, one or more provisos may apply such as: n is 0, 1, or 2; at least three SO₃H are present; at least four SO₃H are present; and two, three, or four SO₃H are present.

Illustrative alkane multi-sulfonic acids include the following: ethane-1,1,2,2-tetrasulfonic acid (CH(SO₃H)₂—CH(SO₃H)₂); ethane-1,1,2-trisulfonic acid (CH₂(SO₃H)—CH(SO₃H)₂); ethane-1,2-disulfonic acid (CH₂(SO₃H)—CH₂(SO₃H)); propane-1,1,2-trisulfonic acid (CH₃—CH(SO₃H)—CH(SO₃H)₂); and butane-1,1,2-trisulfonic acid (CH₃—CH₂—CH(SO₃H)—CH(SO₃H)₂).

The unprotonated form the alkane multi-sulfonic acids (i.e., the alkane multi-sulfonates) are also encompassed. Salts (e.g., alkali salts such as sodium or potassium) of the disclosed alkane multi-sulfonic acids are also encompassed. In embodiments, a salt of the alkane multi-sulfonic acid may be used in place of the alkane multi-sulfonic acid.

Processes for Preparing Alkane Multi-Sulfonic Acids

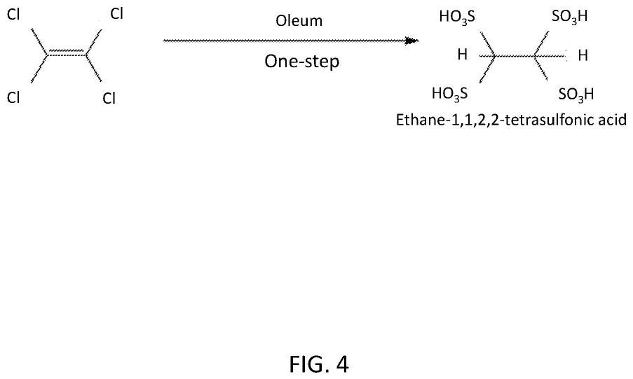

FIG. 4 is a schematic illustrating a one-step process which may be used to prepared the present alkane multi-sulfonic acids. The process is illustrated using tetrachloroethylene, but in general, any haloalkene may be used, depending upon the desired alkane. That is, the haloalkene to be used corresponds to the alkane to be prepared, e.g., 1,2-dichloro-ethylene is a haloalkene which may be used to provide ethane-1,1,2-trisulfonic acid and chloroethylene is a haloalkene which may be used to provide ethane-1,2-disulfonic acid.

By “one-step,” it is meant that the haloalkene is converted to the alkane multi-sulfonic acid(s) in a single synthetic step. This is by contrast to conversion to a salt, followed by a second acidification step to exchange the cation of the salt for a proton/acid. However, this does not preclude additional step(s) to otherwise process or recover the alkane multi-sulfonic acid(s) from the reaction mixture. An embodiment of a one-step process comprises combining the selected haloalkene with oleum or 98% sulfuric acid under conditions to react H₂SO₄ with the carbon-carbon double-bond of the haloalkene. By “oleum,” it is meant sulfuric acid (H₂SO₄) containing sulfur trioxide (SO₃). One or more solvents may be used, but this is not necessary. Thus, an additional advantage of the one-step process is that it may be carried out without using any solvent, i.e., the process is solventless.

The term “conditions” may refer to the amount of SO₃ in the oleum; the use of, or absence of, a solvent(s); the ratio of oleum:haloalkene (or 98% sulfuric acid:haloalkene); the temperature; and the time. In general, values of these parameters may be selected to ensure reaction and to adjust the number of SO₃H groups added to the haloalkene (i.e., tune selectivity to certain alkane multi-sulfonic acids). However, the amount of SO₃ in the oleum may be in a range of from 5% to 60%, from 10% to 50%, or from 20% to 30% (each of these values is equivalent/mole percent) The ratio of oleum:haloalkene (or 98% sulfuric acid:haloalkene) may be in a range of from 1 to 20, from 1 to 15, or from 1 to 10 (each of these values is equivalent/mole ratio). The temperature may be in a range of from 25° C. to 140° C. The time may be in a range of from 5 to 72 hours. If a combination of different types of alkane multi-sulfonic acid(s) are produced, they may be separated from one another, e.g., via distillation. The one-step process is further described in the Examples below, using illustrative halolalkenes and illustrative conditions.

Catalyst Compositions of the Alkane Multi-Sulfonic Acids

Although the present alkane multi-sulfonic acids may be used by themselves in a variety of applications requiring an acid, they may also be combined with other components to form certain catalyst compositions. Illustrative compositions are shown in Table 1, below. More than one type of each component may be used, i.e., more than one type of ionic liquid, more than one type of alkane multi-sulfonic acid, more than one type of Lewis acid, more than one type of base, and/or more than one type of aromatic. In other such embodiments, a single type of each component may be used. Any of the alkane multi-sulfonic acids described above may be used as the “Alkane Multi-Sulfonic Acid” component. A description of each of the remaining components in Table 1 immediately follows.

TABLE 1
Compositions comprising alkane multi-sulfonic acids.

[IL]ₓ-[Alkane Multi-Sulfonic Acid]₍₁₀₀₋ₓ₎
[IL]ₓ-[Alkane Multi-Sulfonic Acid]₍₁₀₀₋ₓ₎-[Aromatic]_{y}
[Lewis Acid]ₓ-[Alkane Multi-Sulfonic Acid]₍₁₀₀₋ₓ₎
[Base]ₓ-[Alkane Multi-Sulfonic Acid]₍₁₀₀₋ₓ₎
[Base]ₓ-[Alkane Multi-Sulfonic Acid]₍₁₀₀₋ₓ₎-[Aromatic]_{y}

Ionic Liquids

Various ionic liquids may be used as a component of a catalyst composition comprising any of the disclosed alkane multi-sulfonic acids. As used in the present disclosure, “ionic liquid” refers to salts composed of at least one cation and at least one anion and are being used in their fluid state. They are generally in their fluid state at or below a temperature of about 100° C.

Representative examples of ionic liquids suitable for use herein are included among those that are described in sources such as J. Chem. Tech. Biotechnol., 68:351-356 (1997); Chem. Ind., 68:249-263 (1996); J. Phys. Condensed Matter, 5: (supp 34B):899-8106 (1993); Chemical and Engineering News, Mar. 30, 1998, 32-37; J. Mater. Chem., 8:2627-2636 (1998); Chem. Rev., 99:2071-2084 (1999); and WO 05/113,702 (and references cited therein), each of which is by this reference incorporated herein for the purpose of the ionic liquids disclosed therein.

Many ionic liquids are formed by reacting a nitrogen-containing heterocyclic ring, preferably a heteroaromatic ring, with an alkylating agent (e.g., an alkyl halide) to form a quaternary ammonium salt, and performing ion exchange or other suitable reactions with various Lewis acids or their conjugate bases to form the ionic liquid. Some ionic liquids are formed by reacting N-, P-, and S-compounds with a Bronsted acid to quaternize the heteroatom. Examples of suitable heteroaromatic rings include substituted pyridines, imidazole, substituted imidazole, pyrrole and substituted pyrroles. These rings can be alkylated with virtually any straight, branched or cyclic C₁₋₂₀ alkyl group, but the alkyl groups are preferably C₁₋₁₆ groups. Various trialkylphosphines, thioethers and cyclic and non-cyclic quaternary ammonium salts may also be used for this purpose. Ionic liquids suitable for use herein may also be synthesized by salt metathesis, by an acid-base neutralization reaction, or by quaternizing a selected nitrogen-containing compound. The synthesis of other ionic liquids suitable for use herein is described in U.S. Pat. No. 8,715,521, which is by this reference incorporated in its entirety as a part hereof for all purposes. Ionic liquids may also be obtained commercially from several companies such as Merck (Darmstadt, Germany), BASF (Mount Olive N.J.), Fluka Chemical Corp. (Milwaukee Wis.), and Sigma-Aldrich (St. Louis Mo.), Iolitec—Ionic Liquids Technologies, GmbH (Heilbronn, Germany), and Proionic (Graz, Austria).

Ionic liquids suitable for use herein comprise a cation and an anion. A variety of cations and anions may be used. Either or both of the ions may be fluorinated. However, in embodiments, neither of the ions are fluorinated. The ionic liquid may include more than one type of cation, more than one type of anion, or both. However, the ionic liquid may include a single type of cation and a single type of anion. When the ionic liquid includes a single type of cation and a single type of anion, however, this does not preclude some amount of ion exchange with other ions in the catalyst composition (derived from other components of the catalyst composition).

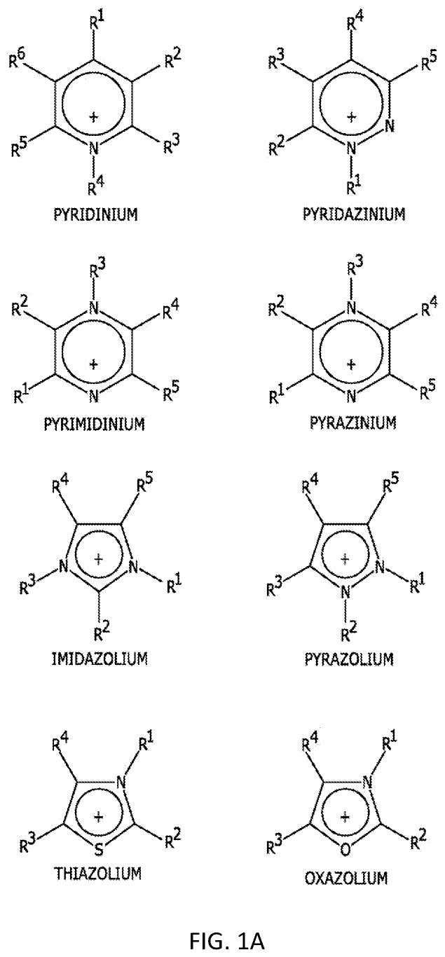

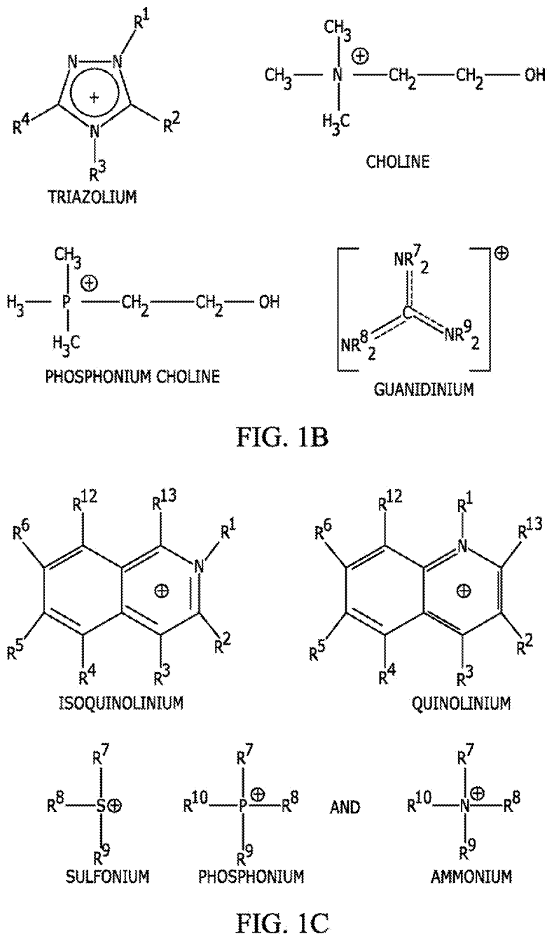

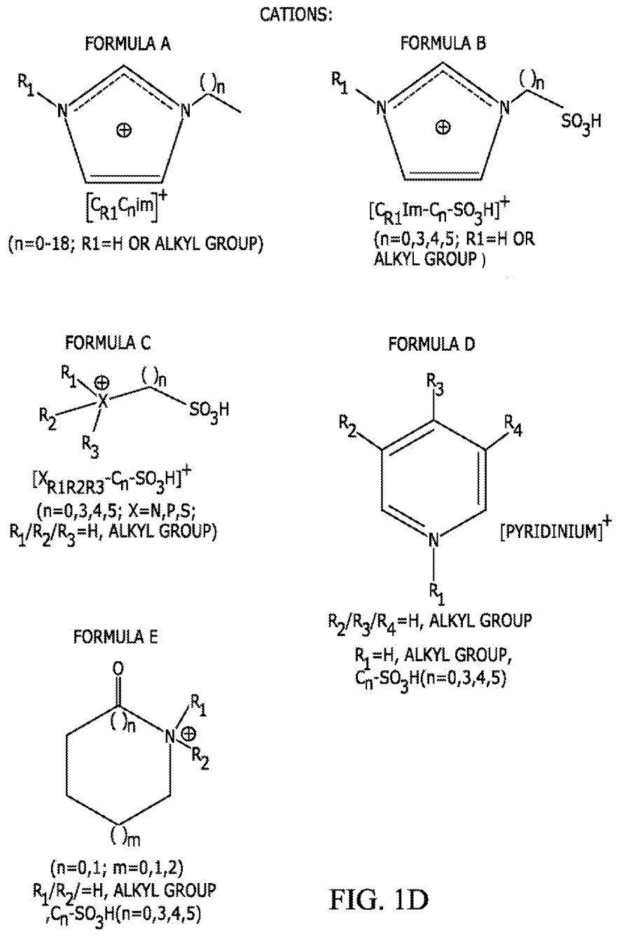

In embodiments, the cation is selected from the group consisting of cations represented by the structures of the formulae shown in FIGS. 1A-1C. In these formulae, the following provisos apply:
(a) R¹, R², R³, R⁴, R⁵, R⁶, R¹² and R¹³ are independently selected from the group consisting of:
(i) H;
(ii) halogen such as F;
(iii) —CH₃, —C₂H₅, or C₃ to C₂₅ straight-chain, branched or cyclic alkane or alkene groups, optionally substituted with at least one member selected from the group consisting of Cl, Br, F, I, OH, NH₂SH, and SO₃H;
(iv) —CH₃, —C₂H₅, or C₃ to C₂₅ straight-chain, branched or cyclic alkane or alkene groups comprising one to three heteroatoms selected from the group consisting of O, N, Si and S, and optionally substituted with at least one member selected from the group consisting of Cl, Br, F, I, OH, NH₂ and SH;
(v) C₆ to C₂₅ unsubstituted aryl, or C₆ to C₂₅ unsubstituted heteroaryl, groups having one to three heteroatoms independently selected from the group consisting of O, N, Si and S, wherein the unsubstituted aryl or unsubstituted heteroaryl may be bonded to the structure via an alkyl (e.g., —CH₂—) spacer group;
(vi) C₆ to C₂₅ substituted aryl, or C₆ to C₂₅ substituted heteroaryl, groups having one to three heteroatoms independently selected from the group consisting of O, N, Si and S; wherein the substituted aryl or substituted heteroaryl may be bonded to the structure via an alkyl (e.g., —CH₂—) spacer group; and wherein said substituted aryl or substituted heteroaryl has one to three substituents independently selected from the group consisting of:
   (A) —CH₃, —C₂H₅, or C₃ to C₂₅ straight-chain, branched or cyclic alkane or alkene groups, optionally substituted with at least one member selected from the group consisting of Cl, Br, F, I, OH, NH₂ and SH,
   (B) OH,
   (C) NH₂, and
   (D) SH; and
(vii) —(CH₂)ₙSi(CH₂)ₘCH₃, —(CH₂)ₙSi(CH₃)₃, —(CH₂)ₙOSi(CH₃)ₘ, where n is independently 1-4 and m is independently 0-4;
(b) R⁷, R⁸, R⁹, and R¹⁰ are independently selected from the group consisting of:
(i) —CH₃, —C₂H₅, or C₃ to C₂₅ straight-chain, branched or cyclic alkane or alkene groups, optionally substituted with at least one member selected from the group consisting of Cl, Br, F, I, OH, NH₂, SH and SO₃H;
(ii) —CH₃, —C₂H₅, or C₃ to C₂₅ straight-chain, branched or cyclic alkane or alkene groups comprising one to three heteroatoms selected from the group consisting of O, N, Si and S, and optionally substituted with at least one member selected from the group consisting of Cl, Br, F, I, OH, NH₂ and SH;
(iii) C₆ to C₂₅ unsubstituted aryl, or C₆ to C₂₅ unsubstituted heteroaryl, groups having one to three heteroatoms independently selected from the group consisting of O, N, Si and S; and
(iv) C₆ to C₂₅ substituted aryl, or C₆ to C₂₅ substituted heteroaryl, groups having one to three heteroatoms independently selected from the group consisting of O, N, Si and S, and wherein the substituted aryl or substituted heteroaryl group has one to three substituents independently selected from the group consisting of:
   (A) —CH₃, —C₂H₅, or C₃ to C₂₅ straight-chain, branched or cyclic alkane or alkene groups, optionally substituted with at least one member selected from the group consisting of Cl, Br, F, I, OH, NH₂ and SH,
   (B) OH,
   (C) NH₂, and
   (D) SH; and
(v) —(CH₂)ₙSi(CH₂)ₘCH₃, —(CH₂)ₙSi(CH₃)₃, —(CH₂)ₙOSi(CH₃)ₘ, where n is independently 1-4 and m is independently 0-4; and
(c) optionally, at least two of R¹, R², R³, R⁴, R⁵, R⁶, R⁷, R⁸, R⁹ and R¹⁰ can together form a cyclic or bicyclic alkyl or alkenyl group.

In embodiments, the ionic liquid comprises a cation selected from one or more members of the group consisting of pyridinium, pyridazinium, pyrimidinium, pyrazinium, imidazolium, pyrazolium, thiazolium, oxazolium, triazolium, phosphonium, ammonium, benzyltrimethylammonium, choline, cholinium, dimethylimidazolium, guanidinium, phosphonium choline, lactam, sulfonium, tetramethylammonium, and tetramethylphosphonium.

In embodiments, the ionic liquid comprises an anion selected from one or more members of the group consisting of: [CH₃CO₂]⁻, [HSO₄]⁻, [CH₃OSO₃]⁻, [C₂H₅OSO₃]⁻, [CH₃C₆H₄SO₃]⁻ ([TSO]⁻), [AlCl₄]⁻, [Al₂Cl₇]⁻, [ZnCl₄]²⁻, [Zn₂Cl₆]²⁻, [Zn₃Cl₈]²⁻, [FeCl₄]⁻, [GaCl₄]⁻, [Ga₂Cl₇]⁻, [InCl₄]⁻, [In₂Cl₇]⁻, [CO₃]²⁻, [HCO₃]⁻, [NO₂]⁻, [NO₃]⁻, [SO₄]²⁻, [PO₃]³⁻, [HPO₃]²⁻, [H₂PO₃]¹⁻, [PO₄]³⁻, [HPO₄]²⁻, [H₂PO₄]⁻, [HSO₃]⁻, [CuCl₂]⁻, Cl⁻, Br⁻, I⁻, SCN⁻, carborates optionally substituted with alkyl or substituted alkyl; carboranes optionally substituted with alkylamine, substituted alkylamine, alkyl or substituted alkyl; and a fluorinated anion.

In embodiments, the ionic liquid comprises an anion selected from one or more members of the group consisting of aminoacetate, ascorbate, benzoate, catecholate, citrate, dimethylphosphate, formate, fumarate, gallate, glycolate, glyoxylate, iminodiacetate, isobutyrate, kojate, lactate, levulinate, oxalate, pivalate, propionate, pyruvate, salicylate, succinamate, succinate, tiglate, tetrafluoroborate, tetrafluoroethanesulfonate, tropolonate, [CH₃CO₂]⁻, [HSO₄]⁻, [CH₃SO₃]⁻, [CH₃OSO₃]⁻, [C₂H₅OSO₃]⁻, [CH₃C₆H₄SO₃]⁻, [AlCl₄]⁻, [Al₂Cl₇]⁻, [ZnCl₄]²⁻, [Zn₂Cl₆]²⁻, [Zn₃Cl₈]²⁻, [FeCl₄]⁻, [GaCl₄]⁻, [Ga₂Cl₇]⁻, [InCl₄]⁻, [In₂Cl₇]⁻, [CO₃]²⁻, [HCO₃]⁻, [NO₂]⁻, [NO₃]⁻, [SO₄]²⁻, [PO₃]³⁻, [HPO₄]²⁻, [H₂PO₄]⁻, [HSO₃]⁻, [CuCl₂]⁻, Cl⁻, Br⁻, I⁻, SCN⁻, [BF₄]⁻, [PF₆]⁻, [SbF₆]⁻, [CF₃SO₃]⁻, [HCF₂CF₂SO₃]⁻, [CF₃HFCCF₂SO₃]⁻, [CHF₂CF₂CF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, [(CF₂HCF₂SO₂)₂N]⁻, [(CF₃CFHCF₂SO₂)₂N]⁻, [N(CN)₂]⁻, F⁻, and anions represented by the structure of the following formula, [R₁₁COO]⁻, wherein R¹¹ is selected from the group consisting of:
(i) —CH₃, —C₂H₅, or C₃ to C₁₀ straight-chain, branched or cyclic alkane or alkene groups, optionally substituted with at least one member selected from the group consisting of Cl, Br, F, I, OH, NH₂ and SH;
(ii) —CH₃, —C₂H₅, or C₃ to C₁₀ straight-chain, branched or cyclic alkane or alkene groups that contain one to three heteroatoms selected from the group consisting of O, N, Si and S, and are optionally substituted with at least one member selected from the group consisting of Cl, Br, F, I, OH, NH₂ and SH;
(iii) C₆ to C₁₀ unsubstituted aryl, or C₆ to C₁₀ unsubstituted heteroaryl, groups having one to three heteroatoms independently selected from the group consisting of O, N, Si and S; and
(iv) C₆ to C₁₀ substituted aryl, or C₆ to C₁₀ substituted heteroaryl, groups having one to three heteroatoms independently selected from the group consisting of O, N, Si and S; and wherein the substituted aryl or substituted heteroaryl group has one to three substituents independently selected from the group consisting of:
   (A) —CH₃, —C₂H₅, or C₃ to C₁₀ straight-chain, branched or cyclic alkane or alkene groups, optionally substituted with at least one member selected from the group consisting of Cl, Br, F, I, OH, NH₂ and SH,
   (B) OH,
   (C) NH₂, and
   (D) SH.

In embodiments, the cation of the ionic liquid is selected from an imidazolium, an ammonium, a phosphonium, a sulfonium, a pyridinium, and a lactam. The cation may be protic or aprotic. The proton in the protic cation may be from a —SO₃H group. Illustrative imidazolium, ammonium, phosphonium, sulfonium, pyridinium, and lactam cations are shown in FIG. 1D. In embodiments, the cation of the ionic liquid is selected from the group consisting of cations represented by the structures of the formulae shown in FIG. 1D, i.e., Formulae A-E. In these formulae, the provisos noted in FIG. 1D apply.

The anion of the ionic liquid may be a sulfonate. The sulfonate may have the formula [R—SO₃]⁻, wherein R is an alkyl group or an aryl group. The alkyl group may be a linear alkyl group in which the number of carbons may range from, e.g., 1 to 12. The alkyl group may be unsubstituted, by which it is meant the alkyl group contains only carbon and hydrogen and no heteroatoms. The alkyl group may be substituted, by which it is meant an unsubstituted alkyl group in which one or more bonds to a carbon(s) or hydrogen(s) are replaced by a bond to non-hydrogen and non-carbon atoms. Non-hydrogen and non-carbon atoms include, e.g., a halogen atom such as F. Aryl groups may be unsubstituted or substituted as described above with respect to alkyl groups. However, substituted aryl groups also refer to an unsubstituted monocyclic aryl group in which one or more carbon atoms are bonded to an alkane. The alkane may be linear, have various numbers of carbon, and may be unsubstituted or substituted as described above with respect to alkyl groups.

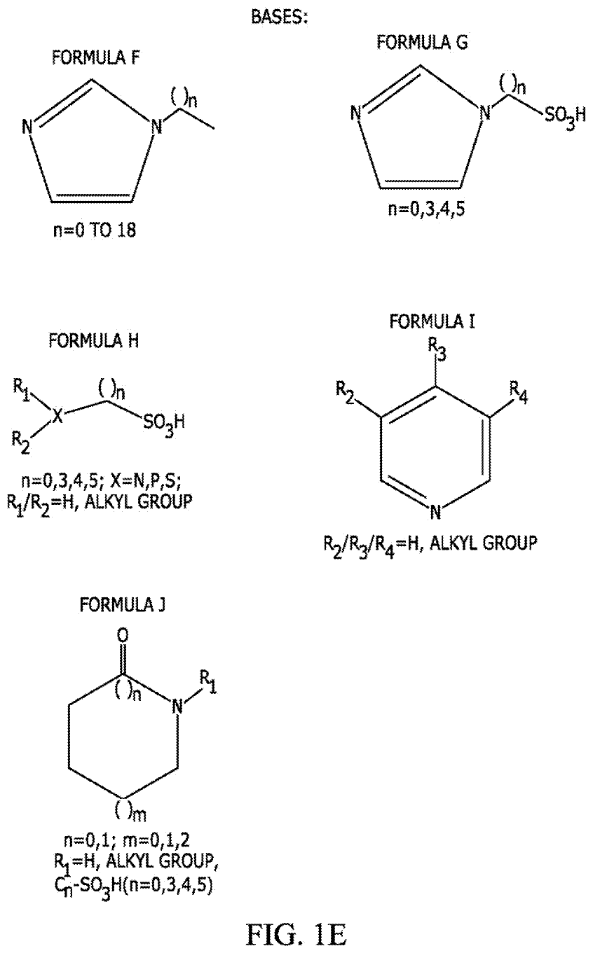

The anion may be a carboxylate. The carboxylate may have the formula [R—CO₂]⁻, wherein R is an alkyl group as described above with respect to sulfonate. This means that fluoroalkane carboxylates are encompassed, e.g., R may be CF₃, HCF₂CF₂, CF₃HFCCF₂, etc. The carboxylate (or fluoroalkane carboxylate) may be a dicarboxylate, a tricarboxylate, a tetracarboxylate, etc. Other anions which may be used include [HSO₄]⁻, dicyanamide; and inorganic anions such as [BF₄]⁻, [PF₆]⁻, and a halide. Illustrative anions are shown in FIG. 2. In [HCF₂(CF₂)ₙSO₃]⁻, n may be 0, 1, 2, or 3.

Ionic liquids disclosed in the following references may also be used: U.S. Pat. Nos. 8,771,626; 8,779,220; 8,808,659; U.S. Pat. Pub. No. 20100331599; U.S. Pat. Nos. 7,432,408; 9,914,674; U.S. Pat. Pub. No. 20160289138; U.S. Pat. Pub. No. 20140113804; U.S. Pat. Pub. No. 20160167034; U.S. Pat. Pub. No. 20150315095; and U.S. Pat. Nos. 9,567,273; 9,346,042; 9,260,668; 9,096,487; 8,692,048; 8,653,318; 8,633,346; 8,569,561; 8,552,243; and 7,285,698. Each of these is by this reference incorporated herein for the purpose of the ionic liquids disclosed therein.

In the ionic liquids, various relative amounts of the cation(s) and anion(s) may be used. In embodiments, the molar ratio of the cation:anion is in the range of from 1:1 to 4:1.

Known methods may be used to prepare ionic liquids. Other ionic liquids may be commercially available.

Aromatics

Various aromatics may be used as a component of a catalyst composition comprising any of the disclosed alkane multi-sulfonic acids, including combinations of different types of aromatics. However, a single type of aromatic may also be used.

The aromatic may be monocyclic having one or more unfused aromatic rings. Each aromatic ring may have various members, e.g., a 5-membered ring, a six-membered ring, etc. Monocyclic aromatics may be unsubstituted, by which it is meant the aromatic contains only carbon and hydrogen and no heteroatoms. Unsubstituted monocyclic aromatics have a single aromatic ring. Monocyclic aromatics may be substituted, by which it is meant an unsubstituted aromatic in which one or more bonds to a carbon(s) or hydrogen(s) are replaced by a bond to non-hydrogen and non-carbon atoms. Non-hydrogen and non-carbon atoms include, e.g., a halogen atom such as F, Cl, Br; 0; N; etc. However, substituted monocyclic aromatics also refer to an unsubstituted monocyclic aromatic in which one or more carbon atoms are bonded to an unsubstituted or substituted alkane or another unsubstituted or substituted monocyclic aromatic. The alkane may be linear or branched, have various numbers of carbon atoms, and may be unsubstituted or substituted. “Unsubstituted” means containing only carbon and hydrogen and no heteroatoms. The alkane group may be substituted, by which it is meant an unsubstituted alkane in which one or more bonds to a carbon(s) or hydrogen(s) are replaced by a bond to non-hydrogen and non-carbon atoms. Non-hydrogen and non-carbon atoms include, e.g., a halogen atom such as F, Cl, Br, and I. Thus, monocyclic aromatics include benzene, biphenyl, triphenyl, furan, pyridine, pyrrole, etc. (each which may be unsubstituted or substituted).

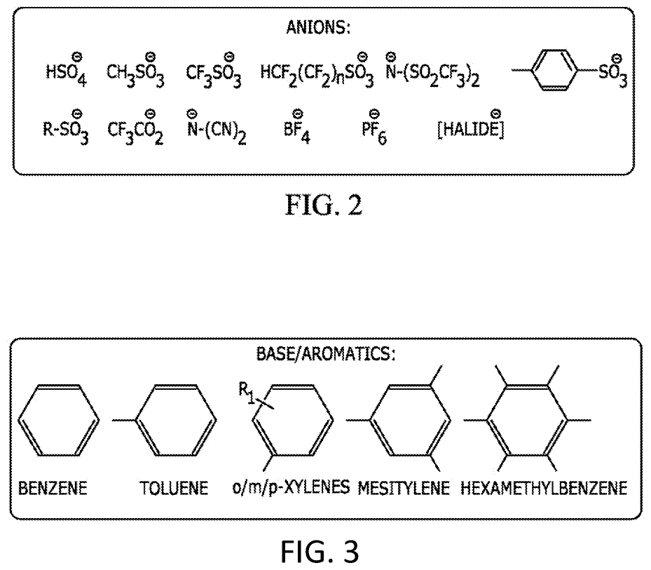

The monocyclic aromatic may have the formula C₆R₆, wherein each R is independently selected from hydrogen, a halogen, and an alkyl group. The alkyl group may be linear or branched have various numbers of carbon atoms and may be unsubstituted or substituted as described above with respect to alkyl groups in “Acids.” Illustrative such monocyclic aromatics are shown in FIG. 3.

Polycyclic aromatics may be used. Polycyclic aromatics have fused aromatic rings (e.g., two, three, etc. rings). Each ring may have various members and may be unsubstituted or substituted as described for monocyclic aromatics. Naphthalene, anthracene, phenanthrene, benzofuran are illustrative polycyclic aromatics.

The aromatic used may be one which forms, in situ, an ionic liquid when combined with the alkane multi-sulfonic acid in forming the catalyst composition.

Lewis Acids

Various Lewis acids may be used as a component of a catalyst composition comprising any of the disclosed alkane multi-sulfonic acids, including combinations of different types of Lewis acids. However, a single type of Lewis acid may also be used. The Lewis acid may be a metal salt. Illustrative metal salts include AlCl₃, ZnCl₂, FeCl₃, GaCl₃, InCl₃, CuCl, and BiCl₃. The Lewis acid may be a metal-containing ionic liquid/salt/liquid coordination complex that behaves as a Lewis acid, including any such ionic liquids described above.

Bases

In embodiments, a base is used which forms, in situ, an ionic liquid when combined with any of the disclosed alkane multi-sulfonic acids. Thus, any base which generates any of the cations described in “Ionic Liquids,” above, upon combination with any of the disclosed alkane multi-sulfonic acids may be used. By way of illustration, the base may be an imidazole, an ammonia, a phosphine, a sulfide, a pyridine, or a lactam. The base be selected from the group of compounds having any of the formulae shown in FIG. 1E, i.e., Formulae F-J. In these formulae, the alkyl group may be as defined above with respect to sulfonate in “Ionic Liquids.” Different types of bases may be used or a single type of base.

As noted above, one or more of any of the disclosed ionic liquids, aromatics, Lewis acids, and bases may be combined with one or more of any of the disclosed alkane multi-sulfonic acids to form a catalyst composition. As also noted above, ion exchange generally occurs between the various components of the catalyst compositions, once formed. In addition, there may be some overlap between compounds suitable for the various components, e.g., some compounds may be suitable as a base and an aromatic. However, catalyst compositions described as comprising, e.g., an “alkane multi-sulfonic acid,” an “ionic liquid,” and an “aromatic” refer to catalyst compositions in which separate and distinct chemicals have been combined to form the catalyst composition regardless of how the various ions may subsequently rearrange/associate therein. For example, a catalyst composition described as comprising an “alkane multi-sulfonic acid,” an “ionic liquid,” and an “aromatic” means that a chemically distinct alkane multi-sulfonic acid, a chemically distinct ionic liquid, and a chemically distinct aromatic were combined to form the catalyst composition. As another example, a catalyst composition described as comprising an alkane multi-sulfonic acid and an ionic liquid refers to compositions in which a chemically distinct alkane multi-sulfonic acid and a chemically distinct ionic liquid were combined to form the catalyst composition.

The particular component or combination of components may be selected to achieve certain behavior in a catalytic conversion reaction, e.g., desired conversion or desired product selectivity. Similarly, the components may be present at various amounts selected to achieve certain behavior.

Referring back to Table 1, in the compositions [IL]x-[Alkane Multi-Sulfonic Acid]₁₀₀₋ₓ, [Lewis Acid]x-[Alkane Multi-Sulfonic Acid]₁₀₀₋ₓ, and [Base]x-[Alkane Multi-Sulfonic Acid]₍₁₀₀₋ₓ₎, the parameter x refers to a weight (wt) %, i.e., ((weight of the ionic liquid/Lewis acid/base)/(combined weight of the ionic liquid/Lewis acid/base and the alkane multi-sulfonic acid))*100. In embodiments, x is in a range of from 0.1 wt % to 90 wt % and the haloalkane sulfonic acid is present at an amount in a range of from 99.9 wt % to 10 wt %. In embodiments, x is in a range of from 2 wt % to 80 wt % and the alkane multi-sulfonic acid is present at an amount in a range of from 98 wt % to 20 wt %. This includes embodiments in which the ionic liquid/Lewis acid/base is present at an amount in a range of from 2 wt % to 80 wt %, from 5 wt % to 60 wt %, from 5 wt % to 30 wt % or 5 wt % to 20 wt % and the alkane multi-sulfonic acid is present at an amount in a range of from 98 wt % to 20 wt %, from 95 wt % to 40 wt %, 95 wt % to 70 wt % or 95 wt % to 80 wt %, respectively.

In the compositions [IL]ₓ[Alkane Multi-Sulfonic Acid]₍₁₀₀₋ₓ₎-[Aromatic]y and [Base]X-[Alkane Multi-Sulfonic Acid]₍₁₀₀₋ₓ₎-[Aromatic]y, x is as defined above and y refers to ((weight of the aromatic)/(combined weight of the ionic liquid/base and alkane multi-sulfonic acid))*100. In embodiments, the aromatic component may be present in any amount up to its saturation point in the composition. In embodiments, y is in a range of from of 0.1 wt % to 25 wt %. This includes from 1 wt % to 15 wt %, 1 wt % to 10 wt %, from 3 wt % to 9 wt %, or from 5 wt % to 8 wt %. In embodiments, y may be in a range of from 0.1 wt % to 100 wt % or from 0.1 wt % to 50 wt %.

An amount of water may be present in the catalyst composition. However, in embodiments, the catalyst composition consists or consists essentially of the components of Table 1.

Other components may be included in the catalyst compositions such as multi-ammonium salts/surfactants described in R. Kore, B. Satpati, R. Srivastava, Synthesis of Dicationic Ionic Liquids and their Application in the Preparation of Hierarchical Zeolite Beta, Chemistry—A European Journal, 17 (2011) 14360-14365 and R. Kore, R. Srivastava, B. Satpati, ZSM-5 zeolite nanosheets with remarkably improved catalytic activity synthesized using a new class of structure directing agents, Chemistry—A European Journal, 20 (2014) 11511-11521, both of which are hereby incorporated by reference in their entirety.

The catalyst compositions may be made by combining the desired components (together or sequentially) at the desired relative amounts. The synthesis may be carried out while stirring and under room temperature.

With regards to catalyst compositions comprising three components, an alkane multi-sulfonic acid, an aromatic, and either an ionic liquid or a base which forms, in situ, an ionic liquid with the alkane multi-sulfonic acid, the following is noted. Without wishing to be bound to any particular theory, it is believed that the three components (or ions generated from the three components) may associate to form a molecular complex having unique, synergistic properties, as distinguished from a simple mixture of the individual components. In the present disclosure, terms such as “ternary complex,” “clathrate,” and the like may be used to describe this molecular complex. However, such terms are not intended to limit the scope of structural form of the molecular complex or catalyst composition. The term “ternary mixture” may also be used in reference to such a catalyst composition. Catalyst compositions comprising two components, e.g., an alkane multi-sulfonic acid and an ionic liquid may be referred to as “binary mixtures.”

Methods of Using the Alkane Multi-Sulfonic Acids and Catalyst Compositions Thereof

The applications for the disclosed alkane multi-sulfonic acids and catalyst compositions thereof are not particularly limited. The alkane multi-sulfonic acids may be used by themselves in a variety of processes requiring an acid. The alkane multi-sulfonic acids may also be used to provide an ionic liquid, e.g., in combination with any of the disclosed bases or aromatics as noted above. Thus, applications requiring an ionic liquid are also encompassed. Finally, any of the disclosed catalyst compositions may be used in a variety of processes requiring an acidic catalyst composition. Illustrative applications are described below.

Alkylation

The present alkane multi-sulfonic acids (and catalyst compositions and ionic liquids formed therefrom, herein after referred to as “related compositions”) may be used in an alkylation process to provide an alkylate product for a motor fuel additive. In embodiments, such a method comprises combining a feedstock and any of the disclosed alkane multi-sulfonic acids/related compositions under conditions to produce the alkylate product. The feedstock may comprise an alkane and an olefin. The alkane may have four or more carbons, i.e., a C4 alkane. The alkane may be an isoalkane. The olefin may have four carbons, i.e., a C4 olefin, but olefins having other numbers of carbons may be used, e.g., C3, C5, C6. The olefin may be an iso-olefin. The feedstock may comprise isobutane and butene, e.g., 2-butene. Other alkanes and olefins may be used, e.g., propane, pentane, propene, isobutene, 1-butene, trans-2-butene, cis-2-butene, pentenes, amylenes, etc. The feedstock may comprise different types of alkanes and different types of olefins. However, a single type of alkane and a single type of olefin may also be used. Under the appropriate conditions, the alkane(s) and olefin(s) of the feedstock are converted into an alkylate product for a motor fuel additive comprising a mixture of branched alkanes. The method may further comprise recovering the alkylate product from the reaction mixture (the combined feedstock and alkane multi-sulfonic acids/related composition).

The conditions under which alkylation occurs refer to parameters such as the amount of the alkane multi-sulfonic acids/related composition used, the amount of feedstock used, the reaction temperature, the reaction time, and the reaction pressure. These parameters may be adjusted to provide desired alkylation behavior, e.g., a desired conversion, C8 selectivity, and T/D ratio.

A variety of reactor systems may be used to carry out the alkylation process, including batch, semi-continuous, continuous, and spray reactor systems.

The present alkane multi-sulfonic acids/related compositions and alkylation reactions may be characterized as being capable of achieving certain properties or results, including a percent conversion, a percent C8 selectivity, and a T/D ratio. Known methods may be used to calculate these values, e.g., see U.S. Pat. Pub. No. 20100331599, which by this reference is incorporated herein in its entirety. In embodiments, the conversion is at least 95%, at least 99%, at least 99.5%, or at least 100%. In embodiments, the C8 selectivity is at least 75%, at least 80%, at least 85%, at least 90%, or at least 98%. In embodiments, the T/D ratio is at least 10, at least 15, at least 20, at least 25, at least 30, or at least 60. These properties may be referenced with respect to a particular set of reaction conditions and may refer to using a pure isobutane and 2-butene feedstock.

The alkylate product formed is also encompassed by the present disclosure. Gasoline comprising the alkylate product is also encompassed.

LAB Process

The present alkane multi-sulfonic acids/related compositions may be used in a process to alkylate benzene. In embodiments, such a process comprises combining benzene, an olefin, and any of the disclosed alkane multi-sulfonic acids/related compositions under conditions to produce a linear alkylbenzene. Under the appropriate conditions, the present alkane multi-sulfonic acids/related compositions can catalyze the addition of the olefin(s) to benzene to provide an alkylbenzene(s). The process may further comprise recovering the alkylbenzene(s) from the reaction mixture. Here, “benzene” refers both to unsubstituted benzene and substituted benzene. Substituted benzene refers to “monocyclic aromatic” as described above in “Aromatics,” except that at least one R is not hydrogen. Substituted benzene also refers to “polycyclic aromatics” as described above in “Aromatics.” The olefin may be a mono-olefin, including a linear alpha olefin. The number of carbon atoms in the olefin may be in the range of from 10 to 13. As with “benzene,” here, “olefin” refers both to unsubstituted olefin and substituted olefin, with the terms “unsubstituted” and “substituted” having meanings analogous to “alkyl” as described above in “Aromatics.” Different types of olefins may be used in the process, i.e., a mixture of different types of olefins.

It is noted that the benzene to be alkylated may itself form a ternary complex with the alkane multi-sulfonic acid and an ionic liquid/base in a catalyst composition used for the alkylation. However, when a catalyst composition is used for the alkylation which comprises any of the disclosed alkane multi-sulfonic acid, an aromatic, and an ionic liquid or a base, the aromatic and the base, if present, are distinct chemical entities from the benzene to be alkylated. This means that either the aromatic/base are different chemical compounds from the benzene to be alkylated (i.e., are not benzene) or are the same chemical compound, but included separately at a separate amount in the catalyst composition.

The conditions under which the alkylation of benzene occurs refer to parameters such as the amount of the alkane multi-sulfonic acids/related compositions, the ratio of benzene:olefin, the reaction temperature, and the reaction time. These parameters may be adjusted to provide, e.g., a desired conversion and/or desired product selectivity. A variety of reactor systems may be used, including batch, semi-continuous, continuous, and spray reactor systems.

The present alkane multi-sulfonic acids/related compositions and alkylation processes may be characterized as being capable of achieving certain properties or results, including a percent conversion and a percent selectivity (for a particular product). Known methods may be used to calculate these values. In embodiments, the conversion is at least 95%, at least 98%, at least 99%, at least 99.5%, at least 99.9% or at least 100%. In embodiments, the selectivity for adding the olefin to benzene at its second carbon (e.g., 2-LAB) is at least 30%, at least 35%, at least 40%, at least 50%, or at least 60%. These properties may be referenced with respect to a particular set of reaction conditions.

Other Applications

Other illustrative applications for the present haloalkane sulfonic acids/related compositions include Beckmann rearrangement reactions, oligomerization reactions, Diels Alder reactions, trans-alkylation of toluene, and Knoevenagel condensation. Conditions which are typically applied when carrying out these reactions may be used.

### EXAMPLES

### Example 1. One-Step Process for Preparing Alkane Multi-Sulfonic Acids

The one-step process to form alkane multi-sulfonic acids is illustrated in FIG. 4. The following Examples are based on this one-step process.

### Example 1-I: Preparation of ethane-1,1,2,2-tetrasulfonic acid [CH(SO₃H)₂—CH(SO₃H)₂] Under Neat Condition

In an N₂-filled glove box, a 350-mL high pressure round bottom flask, equipped with a stir bar, tetrachloroethylene (83 g, 0.50 mol) was reacted with oleum (or fuming sulfuric acid) (5 g, 0.05 mol) under neat condition. After addition, the reaction mixture was heated with magnetic stirring in a temperature-controlled oil bath at 140° C. After 4 days, the flask was removed from oil bath and left to cool on the benchtop. The excess amount of tetrachloroethylene from the reaction mixture was removed by decantation (upper layer) and followed by under high vacuum rota-evaporation, giving a dark brown liquid ethane-1,1,2,2-tetrasulfonic acid [CH(SO₃H)₂—CH(SO₃H)₂] {Purity>80% confirmed by neat NMR (¹H, ¹³C, DEPT-135, DEPT-90, HSQC, and COSY)}.

### Example 1-II: Preparation of ethane-1,1,2,2-tetrasulfonic Acid [CH(SO₃H)₂—CH(SO₃H)₂] Under Neat Condition from Recycled tetrachloroethane

In an N₂-filled glove box, a 350-mL high pressure round bottom flask, equipped with a stir bar, recycled tetrachloroethylene (16.5 g, 0.10 mol) (from experiment in Example 1-I) was reacted with oleum (or fuming sulfuric acid) (1 g, 0.01 mol) under neat condition. After addition, the reaction mixture was heated with magnetic stirring in a temperature-controlled oil bath at 140° C. After 4 days, the flask was removed from oil bath and left to cool on the benchtop. The excess amount of tetrachloroethylene from the reaction mixture was removed by decantation (upper layer) and followed by under high vacuum rota-evaporation, giving a dark brown liquid ethane-1,1,2,2-tetrasulfonic acid (Purity>80% confirmed by neat NMR).

The procedures above may be repeated as follows: the procedure of Example 1-I may use 1,1,2-trichloropropene (0.5 mol) instead of tetrachloroethylene during the synthesis to form propane-1,1,2-trisulfonic acid (CH₃—CH(SO₃H)—CH(SO₃H)₂); and the procedure of Example 1-I may use 1,1,2-trichlorobutene (0.5 mol) instead of tetrachloroethylene during the synthesis to form butane-1,1,2-trisulfonic acid (CH₃—CH₂—CH(SO₃H)—CH(SO₃H)₂).

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. An alkane multi-sulfonic acid or a salt thereof, comprising an alkyl group and at least two sulfonic acid groups, the alkane multi-sulfonic acid having a total number of carbon atoms of from 2 to 9, wherein the alkane multi-sulfonic acid does not comprise a halogen.

2. The alkane multi-sulfonic acid of claim 1 or the salt thereof, wherein the alkyl group is a linear alkyl group, thereby providing a linear alkane multi-sulfonic acid.

3. The alkane multi-sulfonic acid of claim 1 or the salt thereof, having the formula CR₃—CR₂—SO₃H, wherein
at least one R is SO₃H;
each remaining R is independently selected from hydrogen, CₙH₍₂ₙ₊₁₎, CₙH₍₂ₙ₋₁₎, and SO₃H; and
n is 0 to 7.

4. The alkane multi-sulfonic acid of claim 3 or the salt thereof, having the formula CR₂(SO₃H)—CR₂—SO₃H.

5. The alkane multi-sulfonic acid of claim 3 or the salt thereof, wherein the alkane multi-sulfonic acid is a linear alkane sulfonic acid or salt thereof.

6. The alkane multi-sulfonic acid of claim 3 or the salt thereof, having the formula CₙH₍₂ₙ₊₁₎—CR′₂—CR′₂—SO₃H, wherein
n is 0 to 7;
at least one R′ is SO₃H; and
each remaining R′ is independently selected from hydrogen and SO₃H.

7. Then alkane multi-sulfonic acid of claim 6 or the salt thereof, having the formula CₙH₍₂ₙ₊₁₎—CR′(SO₃H)—CR′₂—SO₃H.

8. The alkane multi-sulfonic acid of claim 6 or the salt thereof, wherein the alkane multi-sulfonic acid is a linear alkane sulfonic acid or salt thereof.

9. The alkane multi-sulfonic acid of claim 1 or the salt thereof selected from the group consisting of CH(SO₃H)₂—CH(SO₃H)₂; CH₂(SO₃H)—CH(SO₃H)₂; CH₂(SO₃H)—CH₂(SO₃H); CH₃—CH(SO₃H)—CH(SO₃H)₂; CH₃—CH₂—CH(SO₃H)—CH(SO₃H)₂; salts thereof; and combinations thereof.

10. A composition comprising multiple, different types of alkane multi-sulfonic acids according to claim 1 or salts thereof; or the alkane multi-sulfonic acid according to claim 1 or the salt thereof combined with one or more of an ionic liquid, a Lewis acid, a base, and an aromatic.

11. The composition of claim 10, wherein the composition comprises the ionic liquid.

12. The composition of claim 10, wherein the composition comprises the ionic liquid and the aromatic.

13. A single-step process for preparing the alkane multi-sulfonic acid of claim 1, the process comprising combining a haloalkene with oleum or 98% sulfuric acid under conditions to react H₂SO₄ with the carbon-carbon double-bond of the haloalkene to convert the haloalkene to the alkane multi-sulfonic acid.

14. An alkylation process comprising combining the alkane multi-sulfonic acid of claim 1 or the salt thereof with a feedstock under conditions to produce an alkylate product for a motor fuel additive.

15. An alkylation process comprising combining any the alkane multi-sulfonic acid of claim 1 or the salt thereof with benzene and an olefin or a mixture of olefins under conditions to produce alkylated benzene.
